(19) United States
(12) Patent Application Publication
(10) Pub. No.: US 2006/0029977 A1
(43) Pub. Date: Feb. 9, 2006

(54) Detection and quantification of prion isoforms in neurodegenerative diseases using mass spectrometry

(75) Inventors: Nicholas P. Everett, Meadow Vista, CA (US); James K. Petell, Kennewick, WA (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: ISTA SpA, Milano (IT)

(21) Appl. No.: 10/966,012

(22) Filed: Oct. 15, 2004

Related U.S. Application Data
(63) Continuation of application No. 10/475,234, filed on Oct. 18, 2004.
(60) Provisional application No. 60/284,237, filed on Apr. 17, 2001.
(60) Provisional application No. 60/284,705, filed on Apr. 18, 2001.

(51) Int. Cl. G01N 33/53; C12Q 1/37

(52) U.S. Cl. 435/7.1; 435/23

(57) ABSTRACT

Disclosed are methods, compositions and kits for diagnosing prion-mediated pathological conditions and presence of aberrant prion protein in animal derived products, utilizing mass spectrometry.

### CROSS-REFERENCE TO RELATED APPLICATIONS

This application is a continuation of U.S. application Ser. No. 10/475,234 filed Oct. 17, 2003, which is a national phase entry under 35 U.S.C. §371 of International Application PCT/US02/12012, filed Apr. 17, 2002, published in English, which claims benefit of U.S. Provisional Patent Application 60/284,273, filed Apr. 17, 2001, and U.S. Provisional Patent Application 60/284,705, filed Apr. 18, 2001. The disclosures of all of said applications are incorporated by reference herein.

### TECHNICAL FIELD

The present invention relates to a mass spectrometry based method that provides for the detection or quantitation of aberrant prion isoforms in animals with neurodegenerative diseases and animal-derived products.

### BACKGROUND OF THE INVENTION

Bovine spongiform encephalopathy (BSE) is one of several documented prion neurodegenerative diseases, which includes Creutzfeldt-Jakob disease (CJD) in humans, scrapie in sheep, chronic wasting disease (CWD) in mule deer and elk, transmissible mink encephalopathy (TME), and feline spongiform encephalopathy (FSE) in cats (Aguzzi 2001). Recently, the occurrence of BSE in cows is becoming epidemic in Italy, France, Ireland, Portugal, Germany and other European countries, as it spreads from United Kingdom. Switzerland is second behind the United Kingdom for reported BSE cases. Similar to the transmission of TSE from sheep to cows, it has been reported that genetic evidence exists for the transmission of BSE to humans, as a “new variant” of CJD (nvCJD) (Scott 2000). The nature of the putative transmission to humans is unknown as well as the predisposition of an individual to nvCJD. An unfortunate aspect of TSE is that the prion neurodegenerative diseases are generally latent in onset, which may range from 2-8 years in cows and 3-5 years in sheep after the animal becomes infected. The latent period for humans is believed to be longer than that found in animals. Therefore, the extent of potential horizontal transmission remains largely unknown due to difficulties in the detection of nvCJD until several years after exposure. As expected, since the first reported cases of nvCJD in 1995 it has been rising, mirroring the early growth of BSE cases in the late 1980s. A more severe concern, similar to the AIDs virus, is the potential for rapid transmission of nvCJD through infected blood or tissue donors and bovine based products used in medical treatments and health supplements. Thus there is a pressing need for diagnostic tests that are sufficiently sensitive and reliable to be used to diagnose infected individuals before clinical symptoms develop.

The precise mechanism for the onset of the disease is unknown, however no relationship has been observed between the disease and traditional infectious particles based on nucleic acids (Prusiner 1982a&b; Bolton 1982, Prusiner 1991). Rather, past studies have shown, although not unequivocally, that a specific class of proteins cause infection, denoted prions, and more specifically an aberrant isoform designated PrP^{SC}, can induce the diseased state in laboratory animals and cell cultures. The PrP^{SC} form is distinguishable from the normal cellular its form, denoted PrP^{C}, by its relative resistance to proteases and low solubility. Upon protease treatment of PrP^{SC} protein, the terminal amino acids are truncated leaving a large, resistant core referred to as PrP 27-30, which reflects its observed molecular size in kiloDaltons. It is believed that PrP^{SC} can trigger or act to cascade the conversion of endogenous PrP^{C} into the protease resistant isoform by some unknown mechanism, which accumulates, aggregates and leads to neurodegeneration. The conversion process is thought to facilitate a conformational change of PrP^{C} from an β-helix to β-sheet protein structure.

The clinical aspects of transmissible spongiform encephalopathies are named because of the microscopic or histopathological appearance of large vacuoles in the cortex and cerebellum of the brain in infected animals. The early diagnosis of TSE has been dependent upon the appearance of clinical signs, electroencephalography or invasive methods using brain biopsies. Postmortem histophathological evaluation of ruminant TSEs is based on the appearance of neuronal vacuolation, gliosis and astrocytosis, however these changes may not be realized until the late stages of infection. Other methods using post mortem diagnosis has included the use of immunohistochemical assays to improve the detection of the deposition of prion molecules in brain tissue. A modified method referred to as ID-Lelystad has been performed using immunocytochemistry on thin sections of brain biopsies, which can be completed within 6 hours. The test had 100% correlation with histopathology evaluations, however the method is qualitative and brain samples require the animal to be dead. Further, the nature of the detection protocol is quite laborious and not suitable for robust quantitative analysis.

More recent diagnostic advances have focused on more rapid methods using a variety of other immunological applications that are also less laborious for the detection of TSE, however the single common element that exists with all immunological based assays is the development of a sensitive antibody. The immunological methods currently being used or developed include ELISA or immunometric systems, Western blots and capillary electrophoresis based detection.

The preferred immunometric, or ELISA, quantification utilized an antibody sandwich assay method in conjunction with Protease K treatment to remove the PrP^{C} isoforms (Grassi 2000). This method showed a good correlation with histopathological evaluations. The advantage of this technology is that is suitable for high throughput analysis, but false positives were reported. A modified ELISA employed the use of time-resolved fluorescence immunoassay in conjunction with two concentrations of guanidine hydrochloride to preferentially solubilize one PrP^{C} isoforms relative to the PrP^{SC}(Bamard 2000). The method scores prions in tissues as percentage insoluble prions with the higher ratio being more indicative of aberrant prions. The analysis provides a qualitative rather than a quantitative determination.

A typical Western blot approach involves extracting brain tissue and subsequently subjecting the extract to polyacrylamide gels for separation of proteins followed by immunological probes for detection of prion protein. This type of analysis provides information on the relative molecular size of prion peptides and semi-verification of the result, thereby reducing false positive and negatives. However, polyacrylamide separation of proteins is not robust in determining accurate molecular sizes and has limited sensitivity. Further, the method is only somewhat applicable for low to moderate throughput and is relatively time constraining. In one study, referred to as Prionics Western blotting, it was shown that their results compared favorably to histopathological analysis, a small but significant number of samples tested either false negative (3 of 65) or positive (3 of 263) (Schaller 2000). This method is based on immunocompetition analysis using fluorescently tagged synthetic peptides (Schmerr 1998). Similar to the ELISA method, the sample is first treated with Protease K and subsequently assayed by capillary electrophoresis immunoassay. The study showed greater sensitivity over other methods and was the first method reported using blood samples rather than brain biopsies. The greater sensitivity of the assay facilitated the potential of performing non-invasive blood samples as opposed to biopsies from dead animals. Although this method has greater sensitivity over other immunological methods, it still suffers from the limitation of antibodies raised against a single epitope of a particular prion protein.

The structural differences between the aberrant and native prion isoforms have provided an opportunity for the detection of BSE and other TSEs. Unfortunately, antibodies generated to date have failed to distinguish between the two forms. Thus immunological techniques rely on biochemical pre-protocols that preferentially remove the native isoforms from aberrant prion proteins on the basis of altered solubility or protease stability. Related problems with immunoassays have been the inability to recognize prions across animal species, distinguish between new variants, and have sufficient sensitivity and reliability to be applied to pre-mortem samples.

In the late 1980's two mass spectrometries became available for the analysis of large biomolecules, namely, matrix-assisted laser desorption/ionization (MALDI) time-of-flight mass spectrometry (TOF MS) and electrospray ionization (ESI). Requiring only a minute sample, mass spectrometry provides extremely detailed information about the molecules being analyzed, including high mass accuracy, and is easily automated. Both of these instruments are capable of mass analyzing biomolecules in complex biological solutions. MALDI-TOF MS involves laser pulses focused on a small sample plate comprising analyte molecules embedded in a low molecular weight, UV-absorbing matrix that enhances sample ionization. The matrix facilitates intact desorption and ionization of the sample. The laser pulses transfer energy to the matrix causing an ionization of the analyte molecules, producing a gaseous plume of intact, charged analyte. The ions generated by the laser pulses are accelerated to a fixed kinetic energy by a strong electric field and then pass through an electric field-free region in a vacuum in which the ions travel (drift) with a velocity corresponding to their respective mass-to-charge ratios (m/z). The lighter ions travel through the vacuum region faster than the heavier ions thereby causing a separation. At the end of the electric field-free region, the ions collide with a detector that generates a signal as each set of ions of a particular mass-to-charge ratio strikes the detector. Travel time is proportional to the square root of the mass as defined by the following equation t=(m/(2KE)z)1/2 where t=travel time, s=travel distance, m=mass, KE=kinetic energy, and z=number of charges on an ion. A calibration procedure using a reference standard of known mass can be used to establish an accurate relationship between flight time and the mass-to-charge ratio of the ion. Ions generated by MALDI exhibit a broad energy spread after acceleration in a stationary electric field. Forming ions in a field-free region, and then applying a high voltage pulse after a predetermined time delay (e.g. “delayed extraction™”) to accelerate the ions can minimize this energy spread, which improves resolution and mass accuracy.

In a given assay, 50 to 100 mass spectra resulting from individual laser pulses are summed together to make a single composite mass spectrum with an improved signal-to-noise ratio. The entire process is completed in a matter of microseconds. In an automated apparatus, tens to hundreds of samples can be analyzed per minute. In addition to speed, MALDI-TOF technology has many advantages, which include: 1) mass range—where the mass range is limited by ionization ability, 2) complete mass spectrum can be obtained from a single ionization event (also referred to as multiplexing or parallel detection), 3) compatibility with buffers normally used in biological assays, 4) very high sensitivity; and 5) requires only femtomoles of sample. Thus, the performance of a mass spectrometer is measured by its sensitivity, mass resolution, and mass accuracy.

In order for mass spectrometry to be a useful tool for detecting and quantifying proteins, several basic requirements need to be met. First, targeted proteins to be detected and quantified must be concentrated (e.g., enriched and/or fractionated) in order to minimize the effects of salt ions and other molecular contaminants that reduce the intensity and quality of the mass spectrometric signal to a point where either the signal is undetectable or unreliable, or the mass accuracy and/or resolution is below the value necessary to detect the target protein. Second, mass accuracy and resolution significantly degrade as the mass of the analyte increases. Thus, the size of the target protein or peptide must be within the range of the mass spectrometry device where there is the necessary mass resolution and accuracy. Third, to be able to quantify accurately, one would preferably resolve the masses of the peptides by at least six Daltons to increase quality assurance and to prevent ambiguities. Fourth, the mass spectrometric methods for protein detection and quantification diagnostic screening must be efficient and cost effective in order to screen a large number of samples in as few steps as possible.

Mass spectrometry methods for the quantitation of proteins in complex mixtures have employed a system using protein reactive reagents comprised of three moieties that are linked covalently; an amino acid reactive group, an affinity group and an isotopically tagged linker group (Aebersold et al, 2000). This class of new chemical reagents is referred to as Isotope-Coded Affinity Tags (ICATs) (Gygi et al 1999). The reactive group embodied used sulfhydryl groups that react specifically with the amino acid cysteine. The presence of the affinity group facilitates the isolation of the specifically labeled proteins or peptides from a complex protein mixture. Selected affinity groups include strepavidin or avidin. Only those proteins containing these affinity groups may be isolated. The linker moiety may be isotopically labeled by a variety of isotopes that include ³H, ¹³C, ¹⁵N, ¹⁷O, ¹⁸O and ³⁴S. The use of differential isotopic ICATs provides a method for the quantitation of the relative concentration of peptides in different samples by mass spectrometry. The methods can be used to generate global protein expression profiles in cells and tissues exposed to a variety of conditions.

In an analogous method, the N-terminal amino acids of proteins from two states are differentially labeled using different isotopically tagged nicotinyl-N-hydroxysuccinimide reagents (Munchbach et al, 2000). Unlike the ICAT system, proteins are first separated by two-dimensional SDS polyacrylamide gel electrophoresis before the analysis is performed. The ratio of the isotope for each protein determined by mass spectrometry provides a relative concentration of each protein present in different physiological states.

It is believed that the limitations of mass spectrometry methods employing either ICATs or N-succinylation isotopic tagging are inherently associated with the requirement that the protein from one sample is quantified relative to another state or sample rather than being quantified in absolute amounts. In the case of the ICAT method, it is a requirement that the protein or peptide being quantified contains at least one amino acid that is modified by the reactive group. A related requirement is that the reactive amino acid site on the protein in the two or more states or samples must be equivalently accessible to the reactive group on the ICATs. Similar to antibody methods, if the site is altered or conformationally obscured then the quantitation of the protein will be compromised. An additional limitation in the use of N-succinylation of proteins is that it requires the laborious task of two-dimensional SDS polyacrylamide gel electrophoresis prior to analysis.

There remains a pressing need for easier, more reliable means to rapidly detect, quantify and characterize prion proteins from biological samples particularly complex samples.

### SUMMARY OF THE INVENTION

One aspect of the present invention is directed to a method of detecting a prion-mediated pathological condition in a human or animal, comprising:
(a) obtaining a fluid or cellular or tissue sample from the human or animal;
(b) extracting prion proteins from the sample;
(c) digesting the extracted prion proteins to produce a composition that contains peptide fragments of the extracted prion proteins, wherein the fragments include signature peptides at least one of which is differentially released from an aberrant prion protein compared to a normal prion protein;
(d) analyzing the digested sample and for each signature peptide, a corresponding internal standard peptide, via mass spectrometry; and
(e) generating for each signature peptide, a normalized value obtained by comparing mass spectrometry signals generated by the signature peptides with mass spectrometry signals generated by the corresponding internal standard peptides, wherein a difference between the normalized value for the signature peptide that is differentially released and a normalized value for the signature peptide that is not differentially released, or wherein a difference between the normalized value for the signature peptide that is differentially released and a control, is indicative of a prion-mediated pathological condition.

In some embodiments, the digestion protocol entails treating the sample with a protease, preferably trypsin. In the case of a healthy sample, several signature peptides will be produced, all in roughly equal amounts. If on the other hand, the sample is obtained from a diseased human or animal, the digestion will yield signature prion peptides that are differentially released on account of the fact that the protease resistance of the core region of the disease-related prion protein will reduce the amount of core signature diagnostic peptide detected. Thus, in this case, the differential release is illustrated by a normalized ratio of core signature diagnostic peptides to non-core signature diagnostic peptides that is less than one (1).

In other embodiments, the digestion protocol entails contacting extracted proteins of (b) with a non-specific proteinase under conditions to allow digestion of non-core prion peptides, followed by denaturing non-specific proteinase resistant core prion peptide in the presence of a denaturing agent, followed by contacting denatured core peptide with a protease that is more specific relative to the non-specific proteinase, and wherein in (e) the normalized value for the signature peptide that is differentially released is compared to a control. In this case, digestion of a sample obtained from a healthy or non-diseased animal will not result in the production of statistically significant signature peptide for purposes of the method. In contrast, this digestion of a sample obtained from diseased animal will yield signature peptides that would not otherwise be produced on account of the fact that the chaotropic agent renders the protease-resistant core of the prion protein susceptible to digestion by the specific protease e.g., trypsin. Thus, in this case, signature diagnostic peptides are differentially released and detected from disease-related prion protein because core signature diagnostic peptides from normal prion protein, and non-core signature diagnostic peptides from all prion proteins, will have been previously degraded by the initial treatment with the non-specific protease/proteinase. Thus, in this case, more than one signature peptide is said to be differentially released in that the corresponding peptides from a healthy sample are not present in statistically significant quantity. These two aspects of the invention can be used together to confirm results and thus provide even higher levels of confidence.

Another related aspect of the present invention is directed to a method of detecting a prion-mediated pathological condition in a human or animal, comprising:
(a) obtaining a fluid or cellular or tissue sample from said human or animal;
(b) extracting prion proteins from the sample using a chaotropic agent so as to produce denatured prion proteins;
(c) digesting the denatured prion proteins to produce a composition that contains peptide fragments of the prion proteins, wherein the fragments include signature peptides;
(d) analyzing via mass spectrometry the signature peptides and for each signature peptide, a corresponding internal standard peptide; and
(e) generating for each signature peptide, a normalized value obtained by comparing mass spectrometry signals generated by the signature peptide with mass spectrometry signals generated by the corresponding internal standard peptide, wherein a difference in the normalized value for at least one of the signature peptides compared to a control is indicative of a prion-mediated pathological condition.

In this aspect, extraction with a chaotropic agent and digestion in either a healthy or diseased sample result in production of the same signature prion peptides but each in different amounts when comparing healthy to diseased samples. Denaturing disease-related prion protein allows release of signature diagnostic peptides from the core region that would otherwise be resistant to protease digestion. Thus, in this case, core peptides are differentially released when compared to methods that do not include a denaturing agent.

The mass spectrometry-based methods of the present invention are useful for diagnostic analysis of the family of TSE diseases which includes, but not limited to, Creutzfeldt-Jakob disease (CJD) in humans, BSE (bovine spongiform encephalopathy) in cattle, scrapie in sheep, chronic wasting disease (CWD) in mule deer and elk, transmissible mink encephalopathy (TME), and feline spongiform encephalopathy (FSE) in cats. The intended application of the method can be employed for the monitoring of biological samples that are amenable to non-invasive collection such as serum, saliva, tears, urine, stool, semen, lactation fluid and other biological fluids. The methods provides for the detection and quantitation of prion isoforms, native (PrP^{C}) and aberrant (PrP^{SC}), in uninfected and TSE infected animals.

The mass spectrometry methods of this invention can be used for the improved detection of prion induced neurodegenerative diseases in animals and humans through quantitation and verification of aberrant prion isoforms in sera, body fluids and in tissues samples. They can also be applied to detecting prion proteins in products derived from animals, and not just animals afflicted with a prion-mediated disease. Hence, a further aspect of the present invention is directed to a method of detecting an aberrant prion protein in a product of human or animal origin, comprising:
(a) obtaining a sample from a product of human or animal origin;
(b) extracting prion proteins from the sample;
(c) digesting the extracted prion proteins to produce peptide fragments of the extracted prion proteins, wherein the fragments include signature peptides at least one of which is differentially released from an aberrant prion protein compared to a normal prion protein;
(d) analyzing the peptide fragments and for each of the signature peptides, a corresponding internal standard peptide, via mass spectrometry; and
(e) generating for each signature peptide, a normalized value obtained by comparing mass spectrometry signals generated by the signature peptide with mass spectrometry signals generated by the corresponding internal standard, wherein a difference between the normalized value for the signature peptide that is differentially released and a normalized value for the signature peptide that is not differentially released, or wherein a difference between the normalized value for the signature peptide that is differentially released and a control, is indicative of presence of an aberrant prion protein in the product.

In some embodiments, the digesting entails contacting extracted proteins of (b) with a non-specific proteinase under conditions to allow digestion of non-core prion peptides, followed by denaturing non-specific proteinase resistant core prion peptide in the presence of a denaturing agent, followed by contacting denatured core peptide with a protease, and wherein in (e) the normalized value for the signature peptide that is differentially released is compared to a control.

In a related aspect, the present invention provides a method of detecting an aberrant prion protein in a product of human or animal origin, comprising:
(a) obtaining a sample from a product of human or animal origin;
(b) extracting prion proteins from the sample using a chaotropic agent so as to produce denatured prion proteins;
(c) digesting the denatured prion proteins to produce a composition that contains peptide fragments of the prion proteins, wherein said fragments include signature peptides;
(d) analyzing via mass spectrometry the signature peptides and for each signature peptide, a corresponding internal standard peptide; and
(e) generating for each signature peptide, a normalized value obtained by comparing mass spectrometry signals generated by the signature peptide with mass spectrometry signals generated by the corresponding internal standard peptide, wherein a difference in the normalized value for at least one of the signature peptides compared to a control is indicative of presence of the aberrant prion protein in the product.

The methods can be practiced on any product derived from humans or animals where there is risk of contamination with aberrant prion proteins. In some embodiments the sample is obtained from blood or a blood-derived factor, a commercial food product or ingredient thereof, feed, or cosmetic, nutraceutical or pharmaceutical or an ingredient of said cosmetic, nutraceutical or pharmaceutical.

The present invention provides a relatively sensitive, reliable and verifiable detection and quantitation of diseased prion isoforms in diverse biological samples, with specific applications for non-invasive samples such as sera that may contain significantly lower concentrations of prion molecules. Unlike current immunological based protocols, the present invention does not require the lengthy and laborious production of antibodies, preparation and maintenance of a uniform antibody for kits nor suffer from false positive and negatives as a result of indirect measurement. The described invention provides for multiple, simultaneous, independent, high throughput analyses of the prion proteins, thereby significantly increasing the reliability of the diagnostic results obtained. The mass spectrometry method provides for the verification of prions, which reduces and can even eliminate false positives and negatives, particularly when testing samples that contain low concentrations of prion proteins and/or working near the limits of detection of analytical techniques. The technology is suitable for detection of prion proteins in different species as well as genetic variants that may arise in an animal population, particularly closely related variants. These advantages of the invention compared to existing immunological and other diagnostic methods are summarized in Table 1.

TABLE 1
Comparison of Diagnostic Methods for Prions

| | | Confi- | Through- |
|---|---|---|---|
| Detection Method | Sensitivity | dence | put |
| Immunocytochemistry | ng, qualitative | high | low |
| ELISA (Two-Site) | ng-pg, quantitative | high | high |
| Prionics Western Blot | ng, qualitative | adequate | moderate |
| Capillary Peptide | pg-fg, semi- | adequate | moderate |
| Competition | quantitative | | |
| MS Diagnostics | pg-fg, quantitative | very high | high |

Sensitivity: Order from best to lowest - fg > pg > ng

A yet further aspect of the invention is directed to a kit for the detection or quantification of prion protein in specific sample types. It provides the user with reagents to analyze a particular prion target protein. Thus, in preferred embodiments, the kit contains extraction buffer(s), enrichment resin(s), protease(s), synthetic signature diagnostic peptide(s) and internal standard peptide(s) corresponding to the signature peptide(s), and precise instructions on their use.

### BREIF DESCRIPTION OF THE DRAWINGS

FIG. 1 is a table showing results of a tryptic digestion of bovine prion protein.

FIG. 2 is a table showing results of digestion of bovine prion protein with various proteases.

FIG. 3 is a table showing predicted results of a tryptic digestion of human prion protein.

FIG. 4 is a table showing results of digestion of human prion protein with various proteases.

FIG. 5 is a table showing comparative results of trypsin cleavage peptides for bovine and human prion proteins.

### DETAILED DESCRIPTION

Selection of Diagnostic Peptide Masses for Prions

The basis of the mass spectrometry (MS) method is to measure selected peptides that are diagnostic for the PrP^{SC} isoform. As a diagnostics tool, mass spectrometry does not suffer from the same limitations as immunological protocols. Mass spectrometry operates at the femtomole level of detection that is 10-100 fold greater sensitivity than traditional immunological methods. Further, the uniqueness of each prion signature diagnostic peptide provides a precise “fingerprint” peptide of the prion protein providing very high confidence in analysis.

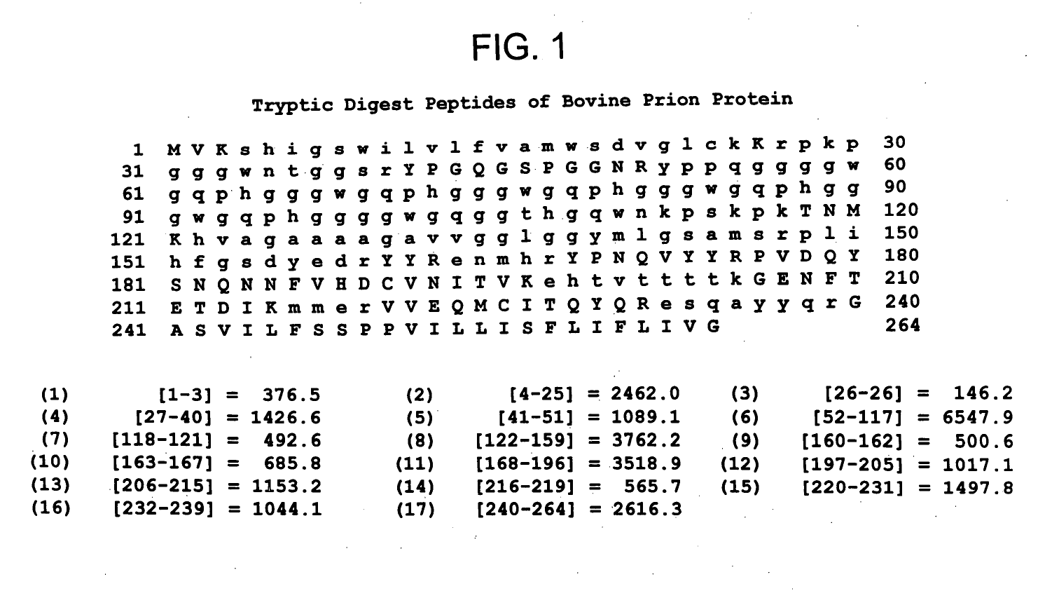

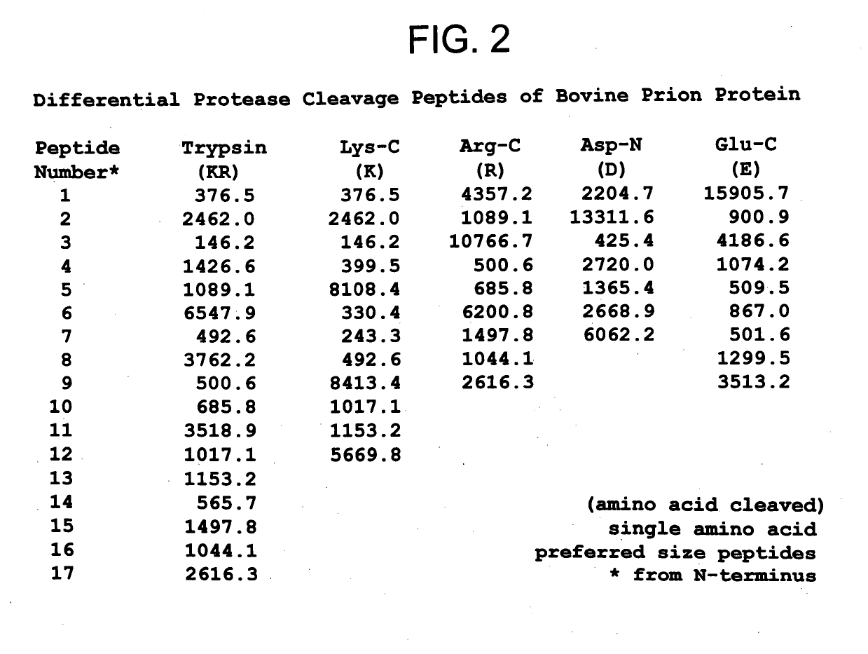

The mass spectrometry method is based on the well documented observation that the PrP^{SC} core is much more resistant to proteases than PrP^{C}. Based on the known sequence of prions, trypsin will cleave bovine PrP^{C} into 16 peptide fragments (the sole single amino acid was omitted) of various molecular sizes ranging from a 146.2 to 6547.9 daltons (See FIGS. 1, 2). Peptides denoted 11, 13 and 17, which contain carbohydrate moieties or the glycosyl phosphatidyl inositol anchor, are considerably larger than the predicted masses based on amino acid sequence alone. In contrast to PrP^{C}, trypsin treatment of PrP^{SC} generates only a restricted number of N-terminal and C-terminal peptides because of the protease resistant core, PrP 27-30. The PK core is comprised of amino acid residues from ˜90 to ˜230. Therefore, at least tryptic peptides 6 through 15 will remain associated with the core.

There are many different types of proteases one skilled in the art may use for cleaving proteins such as endoproteinase-Arg-C, endoproteinase-Aspn-N, endoproteinase-Glu-C (V8), endoproteinase-Lys-C, Factor Xa, papain, pepsin, thermolysin, and trypsin. Chemical compounds, which cleave at specific amino acids (e.g. CNBr which cleaves at methionine residues) can also be used. One skilled in the art will readily recognize that these proteases and chemicals will generate different peptide fragment lengths and thus different peptide masses. It may also be useful to use two or more proteases to enhance the production of desired peptides either sequentially or concurrently. The peptides are preferably in the range from about 900 to 2500 Da but are not limited to these molecular sizes. The peptides generated are said to be derived from the prion protein. The proteolytic step may not be necessary if the targeted proteins can be detected directly by the mass spectrometer with sufficient accuracy to avoid confusion with other non-target proteins.

For example, the cleavage products of bovine prion protein by trypsin-related proteases, Lys-C and Arg-C, produce 11 and 9 peptides, respectively, with only three of each in the 900 to 2500 daltons size range (FIG. 2). Acidic amino acid proteases, Asp-N and Glu-C, which cleave at 6 aspartic and 8 glutamic sites, respectively, generate only 2 and 3 peptides, respectively, that are the preferred size. With a combination of Asp-N and Glu-C, 15 peptides are generated.

Several criteria are used to select which peptide fragments to consider as signature diagnostic peptides. First, the set of peptides needs to include peptides located within and external to the protease resistant core of PrP^{SC}. Second, the peptides are preferably within a size range (MW 900 to 2,500 Da) that is compatible with chemical synthesis and sensitive, accurate detection in the mass spectrometer. For MALDI, the peptides need to be detected under lower laser strength with good spot-to-spot reproducibility and high sensitivity. Third, each internal standard peptide needs to be modified such that the modified peptide mass is not overlapping the native peptide mass (precursor peptide mass) and/or other signature or non-signature diagnostic peptides.

To establish the detection sensitivity, calibration curves for each peptide are constructed using known amounts of the synthetic peptides. Calibration curves are also validated by spiking modified peptides into crude extracts or samples enriched for prion proteins or peptides.

2. Sample Types

The present invention provides mass spectrometric processes for detecting and quantifying prions in a biological sample. Examples of appropriate biological samples for use in the invention include: tissue homogenates (e.g. biopsies); cell homogenates; stool; cell fractions; biological fluids (e.g. urine, serum, semen, cerebrospinal fluid, blood, saliva, amniotic fluid, milk or lactation fluid, mouth wash); and protein-containing products derived from such biological samples or the animals.

Any source of sample protein in a purified or non-purified form which is suspected of carrying a degenerative prion disease can be utilized as starting material for the analysis. The sample can come from a variety of sources. For example: 1) in animal rearing on farms and stockyards, any animal reared for food or clothing production; 2) in food testing the sample can be a commercial food product such as fresh food or processed food (for example infant formula, fresh produce, and packaged food); 3) animal-derived products e.g., blood coagulation factors, animal feed, cosmetics, nutraceuticals and pharmaceuticals; 4) in clinical testing the sample can be human tissue, blood, urine, and infectious diseases; and 5) in domesticated and non-domesticated animals, which include cats, mink rodents, deer, and elk. For clinical analyses, the samples should preferably include tissues or cells that are associated with neurodegenerative prion disease such as brain, spleen, lymphoid organs, spinal cord, kidney, bone marrow or tissue obtained from lymphoreticular system, peripheral or central nervous system, tonsils, the immune system, follicular dendritic cells, lymphocytes and leucocytes.

3. Protein Extraction

Protein can be isolated from a particular biological sample using any of a number of procedures, which are well known in the art, the particular isolation procedure chosen being appropriate for the particular biological sample. For example, soft animal tissues can be homogenized in the presence of appropriate cold buffers in a Waring Blender or polytron or by ultrasonication, and blood cells are easily extracted, after collection by centrifugation, by osmotic lysis or sonication (Current Protocols in Protein Biochemistry, Cold Spring Harbor).

4. Concentration or Enrichment of Target Protein

To obtain an appropriate quantity of a specific protein target on which to perform digestion and then mass spectrometry, concentration (e.g., enrichment) may be necessary. It will be recognized that the enriching step may be accomplished by any number of techniques and methods, which will enrich for the prion protein target. Examples of appropriate means for enrichment include the use of solid support resins (e.g. ion exchangers, affinity gel, and other resins that adsorb proteins). The resins may include beads (e.g. silica gel, controlled pore glass, Sephadex/Sepharose, cellulose, agarose), that can be placed in columns (chromatography, capillary tubes), membranes or microtiter plates (nitrocellulose, polyvinylidenedifluoride, polyethylene, polypropylene), or on flat surfaces or chips or beads placed into pits in flat surfaces such as wafers (e.g. glass fiber filters, glass surfaces, metal surfaces (stainless steel, aluminum, silicon)). Alternatively, the beads may be added batchwise to protein solutions and then removed rapidly by centrifugation, filtration or magnetically (for magnetic beads). Other examples of enrichment include but are not limited to gel electrophoresis, capillary electrophoresis, and pulsed field gel electrophoresis. The choice of method will depend on a number of factors, the amount of protein target present, the physical properties of the protein, the sensitivity required for the detection of the protein and the like.

Resins can separate or absorb targeted proteins based upon the properties of the targeted protein. In this fashion, the targeted protein will either absorb to the resin or contaminating proteins will absorb to the resin. It may be necessary to wash the resin to remove contaminating proteins and thus reduce the complexity of the biological solution. Following a wash step the targeted protein or proteins may be eluted with specific buffers to dissociate the protein. After the proteins have been eluted, the proteins are digested e.g., with a specific protease to generate peptide masses, which are then analyzed by mass spectrometry.

In a preferred embodiment of the present invention, a resin capable of adsorbing, such that the targeted prion protein will be dissociated from contaminating proteins, is used to enrich a prion protein target. A biological sample solution containing proteins is simultaneously enriched and filtered. The amount of sample that can be enriched using a given amount of resin can vary based upon the binding capacity of the resin.

The simultaneous enriching and filtering procedure of the present invention is accomplished using a modified filtration technique. Filter techniques use devices such as filters and rely upon centrifugal or other driving force to wash and elute the sample through a structure such as a membrane. The size of the pores could vary depending upon the protein target and biological sample. It is also conceivable that any ultrafiltration device can be used to practice the present invention where the filter can have a specific molecular weight cut-off. Such filters and ultrafiltration devices are commercially available from Millipore Corp., Bedford, Mass., or LifeScience Purification Technologies, Acton, Mass.

In accordance with various embodiments of the present invention, resin may be placed in a filtration device, for example, using the wells of a microtiter plate. The resin can be added to the microtiter plate in the form of beads. In this embodiment, the resin is added to microtiter wells, which contain a membrane at the bottom of the well through which the sample is allowed to be washed and eluted through the container into a receptacle. The biological sample solution is added to the microtiter plate containing the resin. The sample interacts with the resin and ions in the sample solution are exchanged for ions on the resin. Upon centrifugation or vacuum filtration, the protein targets absorbed to the resin may be washed or eluted off the resin and through the membrane filter. The enriched protein target is then collected from the receptacle.

5. Detection of Peptide Masses by Mass Spectrometry

One skilled in the art will recognize that measurement of the peptide masses of a given prion protein may be accomplished by mass spectrometry. For a general discussion of mass spectrometry and its application to biotechnology see Mass Spectrometry for Biotechnology (1996), ed. Gary Siuzdak, Academic Press (San Diego, Calif.). It will be recognized that, after examining the results of mass spectra from each protein that has been cleaved with a different enzyme, one will need to determine which peptide mass fingerprint best diagnostically distinguishes the target protein.

Diagnostic peptide masses can also be generated for a sequence-independent protein for which the precise amino acid sequence is not known in advance. This is particularly useful if prion variants arise in a population. One skilled in the art will recognize that the order of these peptides in the progenitor protein may not be known, however, it is possible to generate amino acid sequence from the individual peptide masses and compare these with known sequences of other prion proteins. Amino acid sequencing may be accomplished by several means, such as Edman degradation or by post-source decay (PSD) analysis on a mass spectrometry instrument.

The present invention entails the use of internal standard peptides e.g., modified, synthetic peptides that have amino acid identity corresponding to an endogenous prion signature diagnostic peptide, but that are modified to have a characteristic molecular weight e.g., by covalent modification or isotope substitution. The internal standard peptides serve as internal reference standards or calibrants for mass spectrometry analysis. They are used to determine the absolute amount of the prion protein or proteins in a complex mixture. These modified-peptides are of particular use to monitor and quantify the target protein. In this application, the modified peptide is chemically identical to a peptide fragment determined from a signature diagnostic peptide mass fingerprint, except that the peptide has been modified in such a way that there is a distinct mass difference compared to the parent mass that allows it to be independently detected by MS techniques. One skilled in the art can synthesize the amino acid sequence and modify a specific amino acid to distinguish the peptide from the parent peptide. For example, peptides can be modified by acetylation, amidation, anilide, phosphorylation, or modifications where one or more atoms of one or more amino acids can be substituted with a stable isotope to generate one or more substantially chemically identical, but isotopically distinguishable modified-peptides. For example, any hydrogen, carbon, nitrogen, oxygen, or sulfur atoms may be replaced with isotopically stable isotopes: ²H, ¹³C, ¹⁵N, ¹⁷O, or ³⁴S. The modified-peptides can be used in the method described herein to quantify one or several protein targets in a biological sample.

To facilitate mass spectrometric analysis, peptides and proteins generated from either “in-gel” proteolysis or from biological solutions may be concentrated, desalted, and detergents removed from peptide or protein samples by using a solid support. Examples of appropriate solid supports include C₁₈ and C₄ reversed-phase media, ZipTip (Millipore). Immobilization of peptides or proteins can be accomplished, for example, by passing peptides and proteins through the reversed-phase media the peptides and proteins will be adsorbed to the media. The solid support-bound peptides or proteins can be washed and then eluted, which increases overall detection by mass spectrometry.

Preferred mass spectrometer formats for use in the invention are matrix assisted laser desorption ionization (MALDI) and electrospray ionization (ESI). For ESI, the samples, dissolved in water or in a volatile buffer, are injected either continuously or discontinuously into an atmospheric pressure ionization interface (API) and then mass analyzed by a quadrupole. The generation of multiple ion peaks, which can be obtained using ESI mass spectrometry, can increase the accuracy of the mass determination. Even more detailed information on the specific structure can be obtained using an MS/MS quadrupole configuration. The ESI may be connected to a liquid chromatograph (LC, e.g., a micro-LC or nano-LC) into which the digested and signature prion peptides are introduced.

In MALDI mass spectrometry, various mass analyzers can be used, e.g., magnetic sector/magnetic deflection instruments in single or triple quadrupole mode (MS/MS), Fourier transform and time-of-flight (TOF) configurations as is known in the art of mass spectrometry. For the desorption/ionization process, numerous matrix/laser combinations can be used. Ion trap and reflectron configurations can also be employed.

Mass spectrometers are typically calibrated using analytes of known mass. A mass spectrometer can then analyze an analyte of unknown mass with an associated mass accuracy and precision. However, the calibration, and associated mass accuracy and precision, for a given mass spectrometry system can be significantly improved if analytes of known mass are contained within the sample containing the analyte(s) of unknown mass(es). The inclusion of these known mass analytes within the sample is referred to as use of internal calibrants. The preferred practice is to add known quantities of the calibrant. For MALDI-TOF MS, generally only two calibrant molecules are needed for complete calibration, although sometimes three or more calibrants are used. The present invention can be performed with the use of internal calibrants to provide improved mass accuracy.

The invention will be further described by reference to the following experimental work. This section is provided for the purpose of illustration only, and is not intended to be limiting unless otherwise specified. In some of the examples that follow, fetuin is used to illustrate various of the principles of the present invention. Fetuin is a glycoprotein found in bovine and human blood. It has a similar size and carbohydrate moiety to prions and is well characterized and commercially available.

### EXAMPLE 1

### Detection and Quantification of Prions in Bovine Tissue

The purpose of this example of an analysis of a sequence-dependent protein is to detect and quantify diagnostic prion peptides that are diagnostic for the aberrant PrP^{SC} isoforms in cows. The PrP^{SC} core is resistant to proteases while PrP^{C} is not. Based on the known amino acid sequence of the complete bovine prion protein, trypsin cleaves PrP^{C} at lysine and arginine sites into 16 peptides fragments (the sole single amino acid was omitted) of various molecular sizes ranging from 146.2 to 6547.9 daltons (FIGS. 1, 2). Of the 16 peptides, only 7 are of the preferred size and 5 are particularly suitable as candidate signature diagnostic peptides to distinguish between PrP^{C} and PrP^{SC}(Table 2). The cleavage products of prion protein by trypsin related proteases, Lys-C and Arg-C, produce 11 and 9 peptides, respectively, with only three of each in the 900 to 2500 daltons size range (FIG. 2). Acidic amino acid proteases, Asp-N and Glu-C, which cleave at 6 aspartic and 8 glutamic sites, respectively, generate only 2 and 3 peptides, respectively, that are the preferred size. With a combination of Asp-N and Glu-C, 15 peptides are generated. In contrast, trypsin treatment of PrP^{SC} generates a restricted number of N-terminal (4-5 peptides) and C-terminal (1-2 peptides) because of the protease K resistant core, PrP 27-30. The protease resistant core is comprised of amino acid residues from ˜90 to 230. Therefore, at least tryptic peptides 10 through 15 are associated with the core.

TABLE 2

Signature Diagnostic Tryptic Peptides Released
from Bovine Prion Proteins

| | | | |
|---|---|---|---|
| Peptide # | Predicted Mass | Sequence | Residues |

| | | | |
|---|---|---|---|
| 4 | 1426.6 | RPKPGGGWNTGGS(R) | 27-40 |
| 5 | 1089.1 | YPGQGSPGGN(R) | 41-51 |
| 12 | 1017.1 | EHTVTTTT(K) | 197-205 |
| 15 | 1497.8 | VVEQMCITQYQ(R) | 220-231 |
| 16 | 1044.1 | ESQAYYQ(R) | 232-239 |

The detection and quantification of prions is based on the differential sensitivity of the two isoforms, PrP^{SC} and PrP^{C}, to proteases, such as trypsin, and the detection and quantification of a diagnostic set of peptides. To detect and quantify PrP^{SC} in biopsied tissues, samples are extracted using one or more of several extraction methods and protease treatment conditions. The resulting tryptic peptides are analyzed directly by mass spectrometry. The mass spectrometry experiments are carried out on a PerSeptive Biosystems (Framingham, Mass.) Voyager DE-STR equipped with a N² laser (337 nm, 3-nsec pulse width, 20-Hz repetition rate). The mass spectra are acquired in the reflectron mode with delayed extraction. Internal mass calibration is performed with low-mass peptide standards, and mass-measurement accuracy is typically ±0.1 Da. All peptide samples are diluted in a matrix such as α-cyano-4-hydroxycinnamic acid, which has been prepared by dissolving 10 mg in 1 mL of aqueous 50% acetonitrile containing 0.1% trifluoroacetic acid.

(a) Extraction without GdHCl or PK Treatment, Release of Tryptic Diagnostic Peptides

Brain tissues are homogenized using either a hand or polytron homogenizer with a detergent-containing buffer e.g., 150 mM NaCl, 20 mM Tris, pH 7.5 containing 2% sarkosyl (N-lauroylsarcosine). The buffer may also contain a chaotropic agent. After incubation, samples are microcentrifuged for 10 minutes at 13,000×g to remove cellular debris. The pellet is re-extracted, microcentrifuged and the supernatants combined. Before protease digestion, the crude supernatants are spiked with a known amount of acetylated diagnostic peptides to correct for experimental losses and non-specific degradation. For trypsin digestion, duplicate aliquots of the combined, spiked supernatant are digested at 37° C. in a total volume of 25 μL of sequence-grade, modified trypsin (Roche Diagnostics) at a final protein of 25 ng/μL in 25 mM ammonium bicarbonate, pH8.5. After incubation, PMSF is added to inhibit proteases and the incubation mixture is brought to 50% acetonitrile and 0.5% trifluoroacetic acid and clarified by microcentrifugation. All peptide samples are concentrated, desalted, and detergents removed by using either C₄ or C₁₈ reversed-phase ZipTip™ pipette tips as described by the manufacturer (Millipore) and subjected to mass spectrometry analysis as previously discussed.

The amounts of diagnostic tryptic peptides 4, 5, 12, 15 and/or 16 (FIGS. 1, 2, Table 2) are subsequently quantified using synthetic peptides as internal calibrants. The statistical design of the quantification method is based on generating a linear curve between the amount of synthetic peptide and its mass peak using doped samples under mass spectrometry analysis. With the standard curve generated, samples containing known amounts of at least modified synthetic peptides are used to quantify the concentration of related prion peptides in the sample.

The difference in the amount of peptides 12 or 15 and peptides 4, 5 and/or 16 determines the concentration of PrP^{SC}. Peptides 12 and 15 represent only PrP^{C} peptides (Table 3). Therefore, the difference in molar amounts of peptides 12 and 15 to peptides 4, 5 and 16 (after correction for losses and relative sensitivities of detection) reflect the amount of PrPs^{C} present in the samples tested (Table 5).

TABLE 3
Differential Release of Peptides from Prion Isoforms

| | |
|---|---|
| Trypsin | GdHCl/Trypsin |

| Peptide # | PrP^{C} | PrP^{SC} | PrP^{C} | PrP^{SC} |
|---|---|---|---|---|
| N-terminal | | | | |
| 4 | detected | detected | detected | detected |
| 5 | detected | detected | detected | detected |
| Core | | | | |
| 12 | detected | — | detected | detected |
| 15 | detected | — | detected | detected |
| C-terminal | | | | |
| 16 | detected | detected | detected | detected |

(b) Extraction with GdHCl, Release of Tryptic Diagnostic Peptides

It is possible to confirm the concentration of PrP^{SC} by extraction with concentrations of GdHCl or urea, which solublize PrP^{SC}, and subsequent treatment with trypsin. Aliquots of sample homogenates from above are adjusted to 6 M GdHCl and vortexed into solution. After microcentrifugation at 13,000 g for 5 minutes the supernatant is removed and the solution is precipitated with methanol. The precipitate is resuspended in 25 mM ammonium bicarbonate buffer, pH 8.5, containing 3 mM dithiothreitol and either 0.2% SDS or 4 M urea, and then digested with trypsin. For digestion of core PrP^{SC}, duplicate aliquots are digested at 37° C. in a total volume of 25 μL of sequence-grade, modified trypsin (Roche Diagnostics) at a final protein of at least 25 ng/μL in 25 mM ammonium bicarbonate. After incubation, PMSF is added to aliquots to inhibit proteases and calibrant peptides are added in known amounts.

All peptide samples are concentrated, desalted, and detergents removed by using either C₄ or C₁₈ reversed-phase ZipTip™ pipette tips as described by the manufacturer (Millipore) and subjected to mass spectrometry analysis.

The amounts of diagnostic peptides 4, 5, 12, 15 and/or 16 are subsequently quantified using synthetic peptides as internal calibrants. All peptides PrP^{SC} and PrP^{C} peptides are quantified (Table 3). Therefore, the difference in amounts of peptides 12 and 15 detected by this procedure, when compared with the values obtained from procedure (a) above, reflect the amount of PrP^{SC} present in the samples tested (see Table 5).

Digestion with PK, Extraction with GdHCl, Release of Tryptic Diagnostic Peptides

Brain tissues are homogenized using either a hand or polytron homogenizer with 150 mM NaCl, 20 mM Tris, pH 7.5 containing 2% sarkosyl. After incubation, samples are microcentrifuged for 5 minutes at 13,000×g to remove cellular debris For digestion of PrP^{C} and non-core PrP^{SC}, duplicate aliquots are treated with 2 U/ml Protease K at 45° C. for 40 minutes. After addition of PMSF to inhibit Protease K, supernatant aliquots are adjusted to 4 M GdHCl. The solution is precipitated with methanol and the precipitate is resuspended in 25 mM ammonium bicarbonate buffer, pH 8.5, containing 3 mM dithiothreitol and either 0.2% SDS or 4 M urea, and then digested with trypsin. For digestion of core PrP^{SC}, duplicate aliquots are digested at 37° C. in a total volume of 25 μL of sequence-grade, modified trypsin (Roche Diagnostics) at a final protein of 25 ng/mL in 25 mM ammonium bicarbonate.

After incubation, PMSF is added to aliquots to inhibit proteases and calibrant peptides are added in known amounts. The amounts of diagnostic peptides 12 and 15 are subsequently quantified using synthetic peptides as internal calibrants (Tables 4,5). The concentration of PrP^{SC} peptides is directly correlated to the amount of aberrant prion isoforms in biological samples and corresponds to the differences detected in procedure (a) above.

TABLE 4
Preferential Analysis of PrP^{SC} Peptides from PK Treated Samples

Protease K/GdHCl/Trypsin

| Peptide # | PrP^{C} | PrP^{SC} |
|---|---|---|
| N-terminal | | |
| 4 | degraded | degraded |
| 5 | degraded | degraded |
| Core | | |
| 12 | degraded | detected |
| 15 | degraded | detected |
| C-terminal | | |
| 16 | degraded | degraded |

(d) Differential Extraction with GdHCl, no PK Treatment, Release of Tryptic Diagnostic Peptides

As an alternative to (c) above, brain tissues are homogenized using either a hand or polytron homogenizer with 4 volumes of cold 0.1 M Tris buffered saline, pH 7.5 (TBS). Approximately 50 μl aliquots of homogenates are added to an equal amount of a chaotropic agent which in this case was 2 molar guanidine HCl (GdHCl), and vortexed. The concentration of the chaotropic agent may vary e.g., from about 0.5M to about 2M, depending upon the chaotropic agent used. Next, 900 μl of TBS is added, vortexed and microcentrifuged at 13,000×g for 10 minutes. The supernatant is separated from the pellet and discarded. For quantitation of PrP^{SC}, the pellet is suspended in 1001 μl of 6 molar GdHCl and vortexed. Next, 900 μl of TBS is added, vortexed and microcentrifuged at 13,000×g for 10 minutes. The solution is precipitated with methanol and the precipitate is resuspended in 25 mM ammonium bicarbonate buffer, pH 8.5, containing 3 mM dithiothreitol and either 0.2% SDS or 4 M urea, and then digested with trypsin. For digestion of core PrP^{SC}, duplicate aliquots are digested at 37° C. in a total volume of 25 μL of sequence-grade, modified trypsin (Roche Diagnostics) at a final protein of 25 ng/μL in 25 mM ammonium bicarbonate. After incubation, PMSF is added to aliquots to inhibit proteases and calibrant peptides are added in known amounts. All peptide samples were concentrated, desalted, and detergents removed by using either C₄ or C₁₈ reversed-phase ZipTip™ pipette tips as described by the manufacturer (Millipore) and subjected to mass spectrometry analysis.

TABLE 5
Exemplary Results for Healthy and BSE-Infected Samples

| | (a) | | (b) | | (c) | | (d) Differential | |
|---|---|---|---|---|---|---|---|---|
| Peptide | Trypsin | | GdHCl/Trypsin | | PK/GdHCl/Trypsin | | GdHCl/Trypsin | |
| # | Healthy | BSE | Healthy | BSE | Healthy | BSE | Healthy | BSE |
| 4 | + | +++ | + | +++ | − | − | − | ++ |
| 5 | + | +++ | + | +++ | − | − | − | ++ |
| 12 | + | + | + | +++ | − | ++ | − | ++ |
| 15 | + | + | + | +++ | − | ++ | − | ++ |
| 16 | + | +++ | + | +++ | − | − | − | ++ |

The amounts of PrP^{SC} accumulated in BSE-infected samples will vary according to the stage of the disease. The results shown in Table 5 are for BSE samples in which [PrP^{SC}]/[PrP^{total}]=0.66. These data clearly show the opportunity for multiple internal checks of data consistency when using the methods described in this invention

### EXAMPLE 2

### Detection and Quantification of Prions in Human Samples

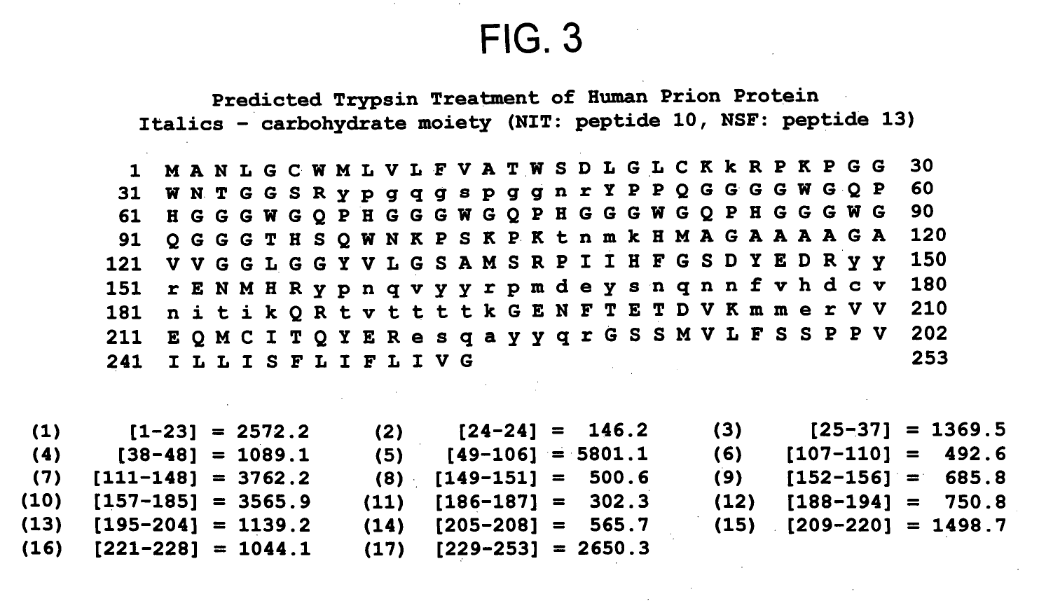

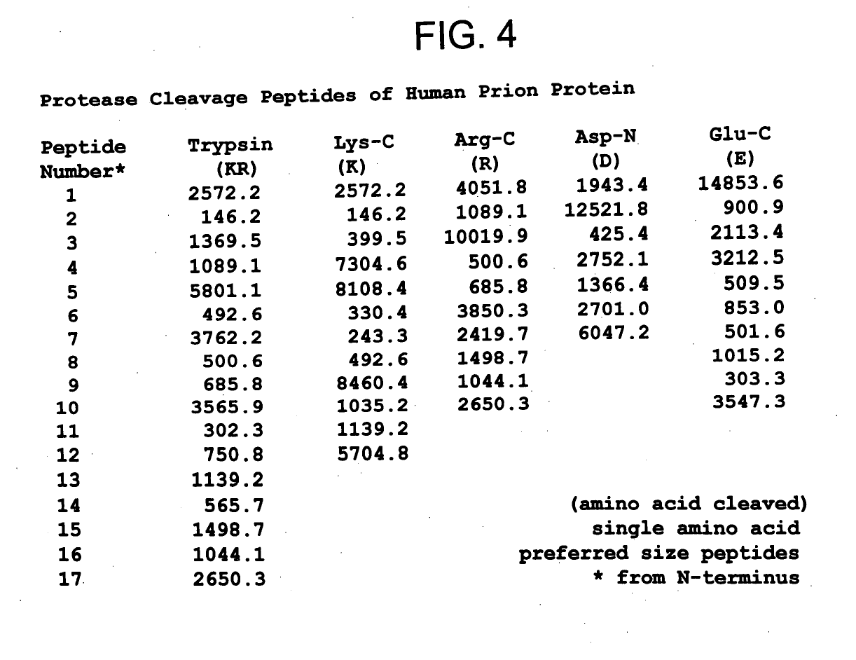

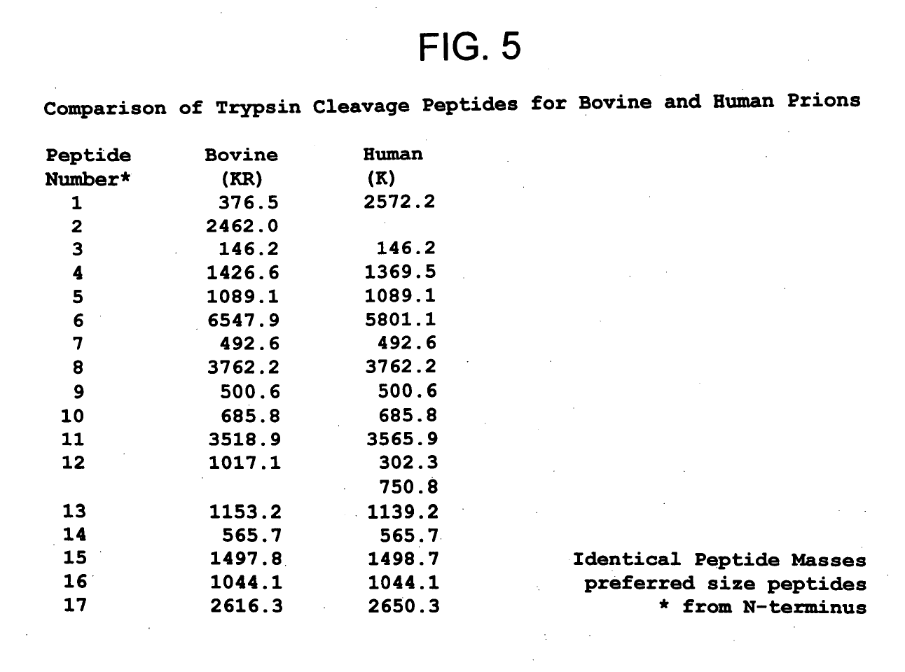

The same invention can also be applied for the detection and quantification of aberrant prions in other animals in which the prion protein has a different amino acid sequence from that of bovine prion protein. In the following example, the human prion protein (novel sequence variant associated with familial encephalopathy (Am. J. Med. Genet. 88:653-56 (1999)) is subjected to protease treatment with a variety of proteases which include endoproteinase-Arg-C(R), endoproteinase-Aspn-N (D), endoproteinase-Glu-C (E), endoproteinase-Lys-C (K), and trypsin (KR). As shown in FIGS. 3 and 4, trypsin treatment of human prion proteins produced 17 peptides of various sizes. Peptides denoted 10 and 13 contain N-linked carbohydrate moieties. Of the 17 trypsin cleavage peptides, 8 peptides are identical molecular size matches to trypsin peptides of bovine prions (FIG. 5). The peptide mass fingerprints constituted by the 8 peptides are suitable for the identification of prions in either bovine or human diseases. Of the 17 trypsin cleavage peptides for human prion, at least 6 peptides are suitable diagnostic markers for the detection of human prions. These diagnostic markers represent the N-terminal, C-terminal and the protease resistant core regions. Additional cleavage peptides, nine peptides in total, are obtained if one uses Arg-C, Asp-N, Lys-C and Glu-C (FIG. 4). The preferred calibrants are selected on the basis of their resolution and sensitivity upon mass spectrometry analysis. The detection and quantitation of aberrant prions in human tissue is performed as described in Example 1, except for the noted differences between signature diagnostic peptides.

### EXAMPLE 3

### Detection and Quantification of Prions in Blood Samples

In this example, blood is collected from the suspected animal or human in EDTA blood tubes to prevent clotting. After collection, samples are centrifuged at 750×g for 30 minutes to obtain a buffy coat. The plasma is removed and stored at −20° C. The buffy coat is collected and re-centrifuged. The pellet is resuspended in phosphate buffered saline (50 mM phosphate, pH 7.0, 150 mM NaCl), sonicated and extracted and analyzed using the methods described in Examples 1 and 2.

In addition to the other enrichment protocols used above, plasma is reacted with Protein A sepharose beads to remove serum IgG. Glycoprotein prions are subsequently enriched by reacting non-bound proteins to lectin chromatography beads that bind glycoproteins. The enriched glycoproteins, with or without elution from the lectin beads, are further processed and analyzed as described previously.

### EXAMPLE 4

### Use of Carbohydrate-Containing Peptides as Diagnostic Markers

For bovine, human and other related animal prion proteins, N-linked carbohydrate moieties are attached to two regions and a third carbohydrate moiety is linked via a lipid attachment region (GPI: glycosylinositol phospholipid). The carbohydrate groups for N-linked chains are known to be heterogeneous, comprising over 30 glycoforms in hamster, and 6 different glycoforms are reported for GPI in the same animal species. The resulting mass heterogeneity of glycosylated peptides would normally limit their consideration as signature diagnostic peptides. However, the presence of carbohydrate chains provide unique opportunities for the isolation, detection and characterization of prion glycoproteins and peptide fragments.

As described in the previous Examples, prion proteins are extracted and subsequently reacted with lectin sepharose sepharose beads for 10 minutes at room temperature. A particular carbohydrate binding resin is wheat germ agglutinin sepharose beads. After microcentrifugation at 13,000×g for 5 minutes, beads are washed with 0.1% Sarkosyl in Tris buffered saline.

Washed beads are treated in a two step process to separate carbohydrate containing peptides from non-carbohydrate peptides. Washed beads are digested overnight at 37° C. in a total volume of 50 μL of sequence-grade, modified trypsin (Roche Diagnostics) at a final protein of 25 ng/μL in 25 mM ammonium bicarbonate. Trypsin is used at approximately 5% per weight to aliquots and digested overnight at 37° C. After incubation, PMSF is added to aliquots to inhibit proteases. Non-glycopeptides are removed by microcentrifugation at 13,000×g for 5 minutes. The supernatant containing the non-glycopeptides are removed and calibrant peptides are added in known amounts. All peptide samples are concentrated, desalted, and detergents removed by using either C₄ or C₁₈ reversed-phase ZipTip™ pipette tips as described by the manufacturer (Millipore) and subjected mass spectrometry analysis. An alternative to the above method is to bind the glycopeptide fragments to lectin beads after the digestion by trypsin or other protease.

To release peptides from the glycopeptides bound to the beads, the beads are treated with N-glycanase (2 units/20 μg of protein) for 2 hours at 37° C. After treatment, the beads are microcentrifuged to separate peptides from bound carbohydrate chains and calibrant peptides are added in known amounts. All peptide samples are concentrated, desalted, and detergents removed by using either C₄ or C₁₈ reversed-phase ZipTip™ pipette tips as described by the manufacturer (Millipore) and subjected to mass spectrometry analysis. This method provides for the enrichment of prion glycopeptides that reside within the core and the GPI peptide. Detection and quantitation of peptides requires a size adjustment for residual N-linked carbohydrate. Recognition of glycopeptide signals in the mass spectrometer is facilitated by comparisons of peptide mass fingerprints of samples before and after treatment with glycanase or glycosidases.

### EXAMPLE 5

### Synthesis and MALDI-TOF Analysis of Prion Signature Diagnostic Peptides and Internal Calibrant Peptides

In this example, five tryptic peptides (RPKPGGGWNTGGSR, YPGQGSPGGNR, EHTVTTTTK, VVEQMCITQYQR, ESQAYYQR) were selected to be synthesized as references for diagnostic peptides, along with their acetylated forms to serve as internal calibrant standards. The peptides were chosen from in silico peptide mass fingerprints of bovine prion protein (Paws software, Proteomics Canada Ltd., www.proteomics.com) to represent both the protease resistant core and non-core regions of the prion protein and to have predicted MH⁺ values between 900 and 2500 (Table 2). A sixth potential peptide from the core region (GENFTETDIK) was not included in the initial chosen set because it includes a site of glycosylation that would increase the peptide mass and represent a special case requiring de-glycosylation. The five peptides were synthesized using standard solid phase methods and the N-terminal of an aliquot of each peptide was modified by N-terminal acetylation (performed by Bruce Kaplan, City of Hope National Medical Center, Pasadena Calif.). Those skilled in the art will appreciate that equivalents, mutants or variants of these peptides, having an amino acid substitution, deletion or addition, could be used.

The purity and veracity of the peptides were checked by HPLC and mass spectrometry. The acetylation modification increased the mass of each peptide by 42 Da. The synthetic peptides were analyzed individually and as mixtures to evaluate detection under low laser strength, spot-to-spot reproducibility and sensitivity of detection. One peptide, EHTVTTTTK, showed a tendency to form adducts with metal ions, generating ions at m/e=1017 (no adduct), m/e=1039 (sodium) and m/e=1066 (potassium), and m/e=1079 if exposed to copper ions. These adducts were greatly reduced by exposure to TFA (trifluoroacetic acid). The formation of metal adduct ions can complicate detection and recognition in the mass spectrometer but can be a useful feature for the enrichment of particular peptides. Analysis of synthetic peptide RPKPGGGWNTGGSR after overnight exposure to trypsin produce a major ion at m/e=1045 (instead of 1426), showing that the adjacent proline residues did not block trypsin digestion at K under the conditions used.

To establish detection sensitivity of potential signature diagnostic peptides, calibration curves were constructed using known amounts of the synthetic peptides. Various concentrations of peptide solutions were prepared and analyzed by MALDI-TOF MS. All peptide samples were diluted in α-cyano-4-hydroxycinnamic acid, which had been prepared by dissolving 10 mg in 1 mL of aqueous 50% acetonitrile containing 0.1% trifluoroacetic acid. Mass spectrometry experiments were carried out on a PerSeptive Biosystems (Framingham, Mass.) Voyager DE-STR equipped with a N₂ laser (337 nm, 3-nsec pulse width, 20-Hz repetition rate). The mass spectra were acquired in the reflectron mode with delayed extraction. Internal mass calibration was performed with low-mass peptide standards, and mass-measurement accuracy was typically ±0.1 Da. All calibration points were examined in triplicate. For example, analysis of synthetic peptide YPGQGSPGGNR in the amount of 0.56, 1.1, 2.2, 4.5 and 9.0 pmol produced peak intensity signals (m/e=1090) of 9800, 17260, 24670, 36485 and 45236, respectively. Analysis of the corresponding acetylated peptide (m/e=1132) produced an equivalent calibration curve. The results demonstrated limits of detection under these conditions in the range 10-100 femtomoles. When used as an internal calibrant standard in protein digests, the signal intensity of the known amount of acetylated signature diagnostic peptide is used to correct for sample-to-sample, day-to-day, and spot-to-

of a 10% homogenate supernatant of bovine muscle and brain tissue to simulate a more complex matrix, were prepared in 25 mM ammonium hydrogen carbonate, total volume 400 μL. Duplicate samples were prepared and applied to aliquots of 50 μL and 250 μL of packed Cibacron resin. In batch processing mode, the samples were incubated by shaking at ambient temperature for two hours, and then microcentrifuged for 2 minutes. Protein analysis using Pierce Coomassie Plus reagent with aliquots of the supernatants indicated that minimal binding had taken place for samples containing only fetuin, and to different extents in the remaining samples. To analyze for fetuin enrichment in the supernatants, sample aliquots with 12 to 159 μg of protein in 100 to 300 μL of supernatant were digested overnight at 37° C. with a each 1.5 μg of sequence-grade, modified trypsin (Roche Diagnostics; www.roche-applied-science.com) in 30 μL of 25 mM ammonium bicarbonate (trypsin is used at at least 1% per weight to the protein). MALDI-TOF MS analysis was carried out as described in Example 5. Digests of resin supernatants of samples containing only fetuin showed the fetuin diagnostic signals m/e 774, 816,1154, 1474, and 2120. In a mixture of fetuin:BSA in a ratio 1:3, only weak signals of 774, 816, and 2120 were observed in the background of BSA digest peptides, while after Cibacron treatment all five of the diagnostic peptides were observed with little background. When a mixture of fetuin:BSA in a ratio 1:30 was analyzed directly, no fetuin signals were observed against the background of BSA digest peptide in the crude mixture, but after Cibacron treatment, the fetuin diagnostic peptides 774, 1474, and 2120 were observed with highly reduced background.

### EXAMPLE 7

### Binding of Denatured Prion Protein to C18 Resin

Reversed phase C18 solid phase extraction material can be used in a wide array of applications to trap, purify, or fractionate proteins and peptides. It is commercially available in bulk, in cartridge format, pipet tip format (Millipore ZipTip™) or 96-well plate format (ANSYS Technologies' SPEC™ SPE products, manufactured with polypropylene plastic and bonded-silica impregnated on a glass fiber disc).

In one example, prion protein from bovine brain homogenates was trapped on Bakerbond SPE™ 7020-06 octadecyl gel (www.vwr.com). The gel was conditioned with methanol and 2% sarcosyl buffer, removed from the SPE columns and used in bulk. Aliquots of 500 uL of settled gel were prepared in 15-mL culture tubes. Up to 0.6 mL of bovine brain tissue homogenates, 10% in homogenization buffer (10 mM NH₄HCO₃, 0.1 M NaCl, 2% sarcosyl), were treated with urea (2.5 mL of 10 M stock solution; for a final concentration of 8 M) and applied to an aliquot of C18 gel. The samples were shaken at room temperature for 5 minutes, centrifuged (2 minutes, approximately 2000 g), and the supernatants analyzed using the Prionics®-Check Western Blot procedure (www.prionics.ch). While crude homogenates gave strong positive results in this assay, no prion protein was detected in the C18-supernatants, showing that all prion protein had bound to the gel.

### EXAMPLE 8

### Binding of Fetuin to Copper-Agarose Resin in the Presence of Bovine Serum Albumin, and On-Resin Trypsin Digestion

Immobilized metal affinity chromatography (IMAC) is a useful method for purifying proteins and peptides based on their affinity for chelated metal ions. Prion protein and serum albumin are known to be copper-binding proteins. For this example, Chelating Sepharose Fast Flow (Amersham-Pharmacia, Cat. No. 17-0575-01, www.apbiotech.com) gel was charged with Cu⁺⁺ ions using 0.2 M CuSO₄. It was then washed with equilibration buffer (below) following the product information, to generate the material that will now be referred to as “Cu⁺⁺-agarose”. Mixtures containing 20 μg of fetuin along with 20, 200, and 2000 μg of BSA, in the presence and absence of 2% sarcosyl, in equlibration buffer (25 mM ammonium bicarbonate, 0.3 M NaCl), total volume 2000 μL, were incubated with aliquots of 400 μL of packed Cu⁺⁺-agarose resin by shaking at ambient temperature for 30 minutes, and then centrifuged for 2 minutes. The supernatants were removed, and the resin samples washed three times with each 2 mL (5 bed volumes) of detergent-free equilibration buffer. Because of the increasing amounts of total protein in the samples, on-resin tryptic digestion experiment were carried out with increasing amounts of modified trypsin (Promega, www.promega.com), at least 0.6 μg trypsin per 100 μg of protein in the sample that was applied to the resin, in 25 mM ammonium bicarbonate (225 μL). For digestion the samples were placed on a shaker, to allow for constant mixing of resin and supernatant overnight at 37° C. The resin samples were then centrifuged, and 50 μL supernatant mixed with 200 μL of 50% acetonitrile/0.5% TFA taken to dryness. Prior to MALDI-TOF analysis, these samples were redissolved in 10 μL of 0.1% TFA in water, and processed using ZipTipT™ if required to optimize signals. On-resin digests of fetuin in the absence of BSA produced signature diagnostic signals at m/e 557, 774, 816 and 1474. On-resin digests of fetuin in the presence of an equal amount of BSA, without detergent, showed the fetuin diagnostic signals m/e 774 and 1474, with 10-fold BSA, only a weak signal for 774 was detected, with 100-fold BSA, no fetuin signal was found in the presence of strong BSA signals. The results for binding in the presence of 2% sarcosyl were comparable. Removal of BSA from extracts (Example 6) before binding prion protein to copper-agarose gel improves the detection of prion signature diagnostic peptides.

### EXAMPLE 9

### Collection of Fetuin on Molecular Sizing Membrane and on-Membrane Digestion with Trypsin

This example demonstrates that bovine fetuin, serving as a model for prion proteins, can be enriched, concentrated, and freed of high concentrations of miscellaneous small molecules (histidine or imidazol from copper agarose immobilized metal affinity chromatography, N-acetyl-D-glucosamine used for elution from WGA lectin, protease inhibitors, detergent, salt) using centrifugal ultrafiltration membrane filters, and that the protein sample can be digested directly on the membrane if desired.

To determine whether small amounts of peptides could be collected after on-membrane digestion or whether they might get adsorbed to the filter, a solution of 25 μg of fetuin in 25 mM NH₄HCO₃ was transferred into a Millipore centrifugal ultrafiltration membrane filter unit with 10,000 molecular weight cutoff range. Sequence-grade, modified trypsin (Roche Diagnostics) in 25 mM ammonium bicarbonate, 2.5 μg/20 μL, was added to the protein on the membrane (final volume 500 μL), the unit vortexed and then transferred to an incubator for digestion overnight at 37° C. After incubation, the unit was centrifuged (20 minutes, 4500 g, IEC Centra GP8R refrigerated centrifuge) and the peptides collected in the flow-through, while any undigested protein and trypsin would remain on the membrane. MALDI-TOF MS analysis was carried out as described in Example 5. The flow-through showed the fetuin diagnostic signals m/e 774, 816,1154, and 1474.

### EXAMPLE 10

### Enrichment of Fetuin on Lectin Resin and Trypsin Digestion

Glycoprotein prions are enriched by reaction to appropriate lectin chromatography beads that show specificity for their oligosaccharide structure, while other proteins remain in the supernatant. Wheat germ agglutinin is reported to react with both prion protein and fetuin.

The lectin wheat germ agglutinin (WGA), covalently bound to agarose gel, was obtained from Sigma (Product No. L1394, labeled with WGA at approximately 6 mg/mL, binding capacity reported as 1-2 mg glycoprotein/mL; www.sigmaaldrich.com). In parallel experiments, 150-μL aliquots of lectin resin were conditioned with pH 7.4 binding buffers (25 mM ammonium bicarbonate and TRIS-HCl) containing 0.1 and 0.5 M NaCl, and with 0.1 M NaCl, with and without 0.1% sarcosyl added. Fetuin samples were adjusted to the same binding buffer concentrations. Aliquots of 80 μg of fetuin in 400 μL of buffer were applied to 150-μL aliquots of packed WGA agarose, and incubated at 4° C. for 3 hours, shaking occasionally. The supernatant was removed, and the gel was washed once with 1 mL of the same buffer to remove unbound protein. Fetuin was eluted using a step gradient from 0.1 M to 0.5 M N-acetylglucosamine in the same buffer/NaCl/sarcosyl solution that was used for the binding step, 500 μL each. In this experiment, trypsin was added directly to the eluates, the digestion carried out over night at 37° C., and samples prepared for MALDI-TOF MS after enrichment of the peptides on ZIPTIP™. The digests showed fetuin diagnostic peaks m/e 774, 816, 1154, 1474, and 2120.

To increase sensitivity, the eluted glycoprotein can be concentrated and salt and N-acetylglucosamine removed using centrifugal ultrafiltration units, 10,000 molecular weight cut-off (Example 9) prior to digestion of the protein. Alternatively, the peptides obtained during the digestion in the presence of salt and N-acetylglucosamine can be purified by HPLC fractionation prior to MALDI-TOF analysis, as described in Example 11.

### EXAMPLE 11

### HPLC Fractionation of Synthetic Prion Peptides

Five synthetic tryptic prion peptides (RPKPGGGWNTGGSR, YPGQGSPGGNR, EHTVTTTTK, VVEQMCITQYQR, ESQAYYQR) from Example 5 were added to a trypic digest of fetuin and subjected to HPLC separation using an Agilent HPLC System, HP1100 series, equipped with a diode array detector. Peptides were monitored at 214 nm. but diode array data over a wider spectral range was also collected. HPLC fractionation was carried out on a Luna C18(2) column, 5 μm, 150×4.6 mm, with a column oven setting of 30° C. Gradient elution was carried out with mobile phase A, 95% water, 5% acetonitrile with 0.1% TFA, and B, acetonitrile with 0.085% TFA, programmed for a gradient from 2 to 35% B in 15 minutes, up to 60% B from 15 to 25 minutes, to 75% B from 25 to 32 minutes, hold at 75% for 3 minutes, back to initial conditions (2% B) from 35 to 36 minutes, hold 2% B until 40 minutes/end of run, at a flow rate of 0.8 mL/min. Fractions were collected in half-minute intervals (400 μL/fraction). Retention times for Prion Signature Diagnostic Peptides number 4, 5, 12, 15, 16 under these conditions were 8.5; 6.8; 5.8; 11.2; and 7.4 minutes, respectively. Aliquots of fractions of interest, 50 μL, were taken to dryness after mixing with 200 μL of 50% acetonitrile/0.5% TFA, for MALDI-TOF MS analysis. In this example, 100 μL of aqueous sample solution containing the digest from 8 μg of fetuin plus 4 mmol of each of the five synthetic prion peptides was injected and fractionated. Table 6 summarizes HPLC and MALDI-TOF MS data obtained for the HPLC profile.

TABLE 6
HPLC-Fractionation of a tryptic digest of fetuin spiked with Prion signature diagnostic
peptides; Detection of peptide masses in HPLC fractions by MALDI-MS

| | | Prion Diagnostic Peptides | | | | | | | | | | | |
|---|---|---|---|---|---|---|---|---|---|---|---|---|---|
| | | #4 | #5 | #12 | #15 | #16 | | | | | | | |
| Retention | | m/z | | | | | Characteristic Fetuin Peptides | | | | | | |
| Time | Fraction | 1425 | | | | | m/z | | | | | | |
| [min] | No. | (1454) | 1089 | 1017 | 1497 | 1044 | 556 | 774 | 816 | 1154 | 1280 | 1474 | 2120 |
| 2.5-3.0 | 6 | | | | | | | | | | | | |
| 3.0-3.5 | 7 | | | | | | | | | | | | |
| 3.5-4.0 | 8 | | | | | | | | | | | | |
| 4.0-4.5 | 9 | | | | | | | | | | | | |
| 4.5-5.0 | 10 | | | | | | | | | | | | |
| 5.0-5.5 | 11 | | | | | | | | | + | | | |
| 5.5-6.0 | 12 | | | + | | | | | | + | | | |
| 6.0-6.5 | 13 | | | + | | | | | | | | | |
| 6.5-7.0 | 14 | | + | | | | | | | | | | |
| 7.0-7.5 | 15 | | + | | | | | | | | | | |
| 7.5-8.0 | 16 | | | | | + | | | | | | | |
| 8.0-8.5 | 17 | | | | | + | | | + | | | | |
| 8.5-9.0 | 18 | + | | | | | | + | + | | | | |
| 9.0-9.5 | 19 | + | | | | | | + | | | | | |
| 9.5-10.0 | 20 | | | | | | | | | | + | | |
| 10.0-10.5 | 21 | | | | | | + | | | | + | | |
| 10.5-11.0 | 22 | | | | | | + | | | | | | |
| 11.0-11.5 | 23 | | | | + | | | | | | | + | |
| 11.5-12.0 | 24 | | | | + | | | | | | | + | + |
| 12.0-12.5 | 25 | | | | + | | | | | | | | |
| 12.5-13.0 | 26 | | | | | | | | | | | | |
| 13.0-13.5 | 27 | | | | | | | | | | | | |
| 13.5-14.0 | 28 | | | | | | | | | | | | |
| 14.0-14.5 | 29 | | | | | | | | | | | | |
| 14.5-15.0 | 30 | | | | | | | | | | | | |

### EXAMPLE 12

### Detection of Abnormal Prion Proteins Using Copper-Agarose Enrichment

Tissue samples (about 5 g) are extracted in 5 mL extraction buffer containing 2% w/v sarkosyl, 0.2M NaCl, protease inhibitor cocktail (Roche Cat. No. 1836170) and 10 mM N-ethylmorpholine (NEMO, Fluka), pH7.4. Aliqots of extract (0.5 mL) are diluted with extraction buffer lacking sarkosyl, 1 mM NEMO, and added to 1.5 mL of copper Sepharose gel (prepared as described in Example 9) and allowed to bind at 25 C for 30 minutes with periodic mixing. The gel is washed (3×3 mL) with extraction buffer lacking sarkosyl and protease inhibitor cocktail before trypsin (Roche Cat. No. 1418033) is added to the gel and incubated at 37C as described in Example 9. Peptides are washed from the gel with either histidine (50 mM) or imidazol (500 mM) in ammonium bicarbonate buffer (3×1.5 mL) before concentration and desalting on ZipTips™ and mass spectrometry analysis with reference to internal calibrant peptides. Samples containing abnormal (infectious) prion protein produce a normalized ratio of core signature diagnostic peptides to non-core signature diagnostic peptides of less than 1.0.

### EXAMPLE 13

### Detection of Abnormal Prion Proteins Using Core Protein Denaturation

Tissue samples (about 5 g) are extracted in 5 mL extraction buffer containing 2% w/v sarkosyl, 0.2M NaCl, protease inhibitor cocktail (Roche Cat. No. 1836170) and 10 mM N-ethylmorpholine (NEMO, Fluka, www.sigmaaldrich.com)), pH7.4. Aliquots of extract (0.5 mL) are added to 10 M urea (2.5 mL) to denature prion proteins and then bound to C-18 resin to concentrate the proteins and permit washing (4×3 mL) with ammonium bicarbonate buffer (25 mM) containing 0.1% sarkosyl. The proteins are eluted from the C-18 resin with acetonitrile (50% v/v) and digested with trypsin. The peptides are analyzed by mass spectrometry and quantitated with reference to internal calibrant peptides. The normalized ratio of core signature diagnostic peptides to non-core signature diagnostic peptides will be approximately 1.0 for both normal and abnormal prion proteins. Samples containing abnormal prions produce a higher concentration of core signature diagnostic peptides by this method compared to the normalized concentration of core diagnostic peptides detected for the same sample by the method described in Example 12.

### EXAMPLE 14

### Detection of Abnormal Prion Proteins Using Proteinase K and Core Protein Denaturation

Tissue samples (about 5 g) are extracted in 5 mL extraction buffer containing 2% w/v sarkosyl, 0.2M NaCl, and 10 mM N-ethylmorpholine (NEMO, Fluka), pH7.4.Aliquots of extract (0.5 mL) are incubated with proteinase K (Roche Product No. 1413783) for 40 minutes at 47 C to digest protease sensitive proteins, including the non-core region of abnormal prion protein, but leaving the prion core region of abnormal prion protein intact. At the end of the proteinase K digestion, Pefabloc SC (Sigma Cat. No. 76307; www.sigmaaldrich.com) or PMSF is added to irreversibly inhibit the proteinase K, and the sample is diluted with 10M urea to a final concentration of 8M urea. The denatured prion core protein is then bound to C-18 resin to concentrate the proteins and permit washing (4×3 mL) with ammonium bicarbonate buffer (25 mM) containing 0.1% sarkosyl. The proteins are eluted from the C-18 resin with acetonitrile (50% v/v) and digested with trypsin. The peptides are analyzed by mass spectrometry and quantitated with reference to internal calibrant peptides corresponding to core signature diagnostic peptides. Only samples containing abnormal prion protein should generate significant amounts of core signature diagnostic peptides. The ratio of normalized core signature diagnostic peptides from this protocol to normalized core signature diagnostic peptides from Example 12 is diagnostic for the presence of abnormal prion protein from an infectious source.

### INDUSTRIAL APPLICABILITY

The present invention has applicability in human and veterinary medicine, particularly from the standpoint of diagnosis of disease, as well as in quality control for detection of prion isoforms in animal-derived products.

### REFERENCED PUBLICATIONS

Aguzzi, A. Monirasio. F. Kaeser, P. S., Nature Rev. 2:118-126 (2001).
Barnard, G. Helmick, B. Madden, S. Gilboume, C. Patel, R., Luminescence 15: 357-362 (2000).
Bolton, D C, McKinley, M P, Prusiner, S., Science 218:1309-1311(1982).
Grassi, J., Creminon, C., Frobert, Y., Fretier, P., Turbica, I., Rezaei, H., Hunsmann,
G., Comoy, E., Deslys, J-P., Arch Virol 16:197-205 (2000).
Prusiner, S. BoRon, D C, Groth, D F, Bowman, K A, Cochran, S P, McKinley, M. P., Biochem. 21:6942-6950 (1982a).
Prusiner, S., Science 216:136-144 (1982b).
Prusiner, S., Science 252:1515-1522 (1991).
Schaller, O, Fatzer, R, Stack, M, Clark, I, Cooley, W, Biffiger, K, Egli, S, Doherr,
Vandevelde, M, Heim, D, Oesch, B, Moser, M., Acta Neuropathol. 98:437-443 (1999).
Schmerr, M J, Allen, J., Electrophor. 19:409-414 (1998).
Scott, M R, Will, R, Ironside, J, Nguyen, H-O B, Tremblay, P, DeArmond, S J, Prusiner, S. B., Proc. Natl. Acad. Sci. USA 96:15137-15142 (2000).
Aebersold, R., Rist, B., Gygi, S. P., Ann. N. Y. Acad. Sci. 919:33-47 (2000).
Gygi, S. P., Rist, B., Gerber, S. A., Turecek, F., Gelb, M., and Aebersold, R., Nat. Biotech. 17(10):994-999 (1999).
Munchbach, M., Quodroni, M., Miotto, G., and James, P., Anal. Chem. 72(17):4047-57 (2000).

All patent and non-patent publications cited in this specification (including web sites) are indicative of the level of skill of those skilled in the art to which this invention pertains. All these publications and patent applications are herein incorporated by reference to the same extent as if each individual publication or patent application was specifically and individually indicated as being incorporated by reference herein. In addition, the entirety of commonly owned international application no. PCT/US02/12014, entitled “METHODS FOR MASS SPECTROMETRY DETECTION AND QUANTIFICATION OF SPECIFIC TARGET PROTEINS IN COMPLEX BIOLOGICAL SAMPLES,” filed of Apr. 17, 2001, is also incorporated herein by reference.

Those skilled in the art will recognize, or be able to ascertain, using no more than routine experimentation, numerous equivalents to the specific substances and procedures described herein. Such equivalents are considered to be within the scope of this invention.

1. A method of detecting a prion-mediated pathological condition in a human or animal, comprising:
(a) obtaining a fluid or cellular or tissue sample from the human or animal;
(b) extracting prion proteins from the sample;
(c) digesting the extracted prion proteins to produce a composition that contains peptide fragments of the extracted prion proteins, wherein the fragments include signature peptides at least one of which is differentially released from an aberrant prion protein compared to a normal prion protein;
(d) analyzing the digested sample via mass spectrometry wherein the digested sample also contains for each signature peptide, a corresponding internal standard peptide; and
(e) generating for each signature peptide, a normalized value obtained by comparing mass spectrometry signals generated by the signature peptides with mass spectrometry signals generated by the corresponding internal standard peptides, wherein a difference between the normalized value for the signature peptide that is differentially released and a normalized value for the signature peptide that is not differentially released, or wherein a difference between the normalized value for the signature peptide that is differentially released and a control, is indicative of a prion-mediated pathological condition.

2. The method of claim 1 wherein the control comprises a normalized value obtained by comparing mass spectrometry signals generated by signature peptides obtained from a healthy human or animal, compared to the corresponding internal standard peptides.

3. A method of detecting a prion-mediated pathological condition in a human or animal, comprising:
(a) obtaining a fluid or cellular or tissue sample from said human or animal;
(b) extracting prion proteins from the sample using a chaotropic agent so as to produce denatured prion proteins;
(c) digesting the denatured prion proteins to produce a composition that contains peptide fragments of the prion proteins, wherein the fragments include signature peptides;
(d) analyzing via mass spectrometry the signature peptides and for each signature peptide, a corresponding internal standard peptide; and
(e) generating for each signature peptide, a normalized value obtained by comparing mass spectrometry signals generated by the signature peptide with mass spectrometry signals generated by the corresponding internal standard peptide, wherein a difference in the normalized value for at least one of the signature peptides compared to a control is indicative of a prion-mediated pathological condition.

4. The method of claim 3 wherein the control comprises a normalized value obtained by comparing mass spectrometry signals generated by the signature peptide obtained from a healthy human or animal, compared to the corresponding internal standard peptide.

5. The method of claim 1 wherein the sample is a fluid sample obtained from serum, cerebrospinal fluid, blood, saliva, tears, urine, semen, amniotic fluid, milk or lactation fluid.

6. The method of claim 1 wherein the sample is a cellular or tissue sample obtained from muscle, skin, eyelids, brain, spinal cord, lymphoid organs, spleen, kidney, bone marrow or tissue obtained from lymphoreticular system, peripheral nervous system, central nervous system, immune system, follicular dendritic cells, lymphocytes or leucocytes.

7. The method of claim 1 wherein said extracting comprises contacting said sample with a buffer.

8. The method of claim 7 wherein said buffer comprises a detergent.

9. The method of claim 8 wherein said detergent comprises SDS or sarkosyl.

10. The method of claim 2 wherein said digesting comprises (c1) contacting extracted proteins of (b) with a non-specific proteinase under conditions to allow digestion of non-core prion peptides, followed by (c2) denaturing non-specific proteinase resistant core prion peptide in the presence of a denaturing agent, followed by (c3) contacting denatured core peptide with a protease, and wherein in (e) the normalized value for the signature peptide that is differentially released is compared to a control.

11. The method of claim 10 wherein the control comprises a normalized value obtained by comparing mass spectrometry signals generated by the signature peptide that is differentially released and contained in a sample obtained from healthy humans or animals, compared to the corresponding internal standard.

12. The method of claim 10 wherein the denaturing agent comprises guanidine hydrochloride, acetonitrile, urea or heat.

13. The method of claim 11 wherein the denaturing agent comprises guanidine hydrochloride in a concentration of from about 4 to about 6M.

14. The method of claim 11 wherein the denaturing agent comprises urea in a concentration of from about 4 to about 8M.

15. The method of claim 1 further comprising (f) concentrating the extracted prior proteins of (b), and wherein said digesting comprises producing peptide fragments of the extracted and concentrated prion proteins.

16. The method of claim 15 wherein said concentrating comprises contacting the extracted proteins of (b) with a resin that adsorbs prion proteins or non-prion proteins.

17. The method of claim 16 wherein said concentrating further comprises filtering the extracted proteins of (b).

18. The method of claim 1 wherein said digesting comprises treating the extracted prion proteins with at least one protease.

19. The method of claim 18 wherein the protease comprises trypsin.

20. The method of claim 1 wherein the composition further comprises a matrix and said analyzing comprises introducing the composition into a matrix assisted laser desorption ionization (MALDI) time-of-flight (TOF) analyzer.

21. The method of claim 20 wherein the matrix comprises alpha-cyano-4-hydroxycinnamic acid.

22. The method of claim 1 wherein said analyzing comprises introducing the composition into an ion trap electrospray ionization apparatus (ESI).

23. The method of claim 1 further comprising (g) introducing the composition into a liquid chromatograph (LC) prior to said analyzing.

24. The method of claim 23 wherein the LC is a micro-LC.

25. The method of claim 23 wherein the LC is a nano-LC.

26. The method of claim 23 wherein said analyzing comprises introducing the composition into an ion-trap ESI.

27. The method of claim 23 wherein said analyzing comprises introducing the composition into a MALDI-TOF analyzer.

28. The method of claim 1 wherein said digesting comprises treating the extracted prion proteins with trypsin, wherein the signature peptides comprise at least one core signature peptide and at least one non-core signature peptide, wherein the internal standard peptides comprises mass-labeled reference peptides, and wherein said generating comprises detecting increased or decreased presence or amount of the core signature peptide relative to the non-core signature peptide.

29. The method of claim 28 wherein the sample is obtained from a bovine, and wherein the core signature peptides comprise peptides EHTVTTTTK (residues 197-205 of SEQ ID NO: 1), GENFTETDIK (SEQ ID NO: 3) or VVEQMCITQYQR (residues 220-231 of SEQ ID NO: 1), or an equivalent, mutant or variant thereof having an amino acid substitution, deletion or addition, and the non-core signature peptides comprise RPKPGGGWNTGGSR (residues 27-40 of SEQ ID NO: 1), PGGWNTGGSR (residues 30-40 of SEQ ID NO: 1), YPGQGSPGGNR (residues 41-51 of SEQ ID NO: 1) or ESQAYYQR (residues 232-239 of SEQ ID NO: 1), or an equivalent, mutant or variant thereof having an amino acid substitution, deletion or addition.

30. The method of claim 28 wherein the sample is obtained from a human, and wherein the core signature peptide comprises peptides QRTVTTTTK (residues 186-194 SEQ ID NO: 2), GENFTETDVK (residues 195-204 of SEQ ID NO: 2) or VVEQMCITQYER (residues 209-220 of SEQ ID NO: 2), or an equivalent, mutant or variant thereof having an amino acid substitution, deletion or addition, and the non-core signature peptide comprises RPKPGGGWNTGGSR (residues 25-37 of SEQ ID NO: 2), PGGWNTGGSR (residues 28-37 of SEQ ID NO: 2), YPGQGSPGGNR (residues 38-48 of SEQ ID NO: 2) or ESQAYYQR (residues 221-228 of SEQ ID NO: 2), or an equivalent, mutant or variant thereof having an amino acid substitution, deletion or addition.

31. The method of claim 28 wherein the signature peptides comprise more than one core prion protein and more than one non-core prion protein.

32. The method of claim 1 wherein the prion-mediated pathological condition is transmissible spongiform encephalopathy (TSE), Creutzfeldt-Jakob disease (CJD), bovine spongiform encephalopathy, scrapie, chronic wasting disease (CWD), transmissible mink encephalopathy (TME), or feline spongiform encephalopathy (FSE).

33. The method of claim 1 wherein the sample of (a) is a first portion of the sample, and wherein said method further comprises:
(f) extracting the prion proteins from a second portion of the sample using a chaotropic agent so as to produce the prion proteins in denatured form;
(g) digesting the denatured prion proteins to produce peptide fragments of the denatured prion proteins, wherein the fragments include signature peptides of the denatured and digested prion proteins;
(h) analyzing via mass spectrometry the signature peptides of (g) and for each signature peptide, a corresponding internal standard peptide; and
(i) generating for each signature peptide, a normalized value obtained by comparing mass spectrometry signals generated by the signature peptide with mass spectrometry signals generated by the corresponding internal standard peptide, wherein a difference in the normalized value for at least one of the signature peptides compared to a control is indicative of a prion-mediated pathological condition; and
(j) comparing indication obtained from (h) with indication obtained from (e).

34. A method of detecting an aberrant prion protein in a product of human or animal origin, comprising:
(a) obtaining a sample from a product of human or animal origin;
(b) extracting prion proteins from the sample;
(c) digesting the extracted prion proteins to produce peptide fragments of the extracted prion proteins, wherein the fragments include signature peptides at least one of which is differentially released from an aberrant prion protein compared to a normal prion protein;
(d) analyzing the peptide fragments and for each of the signature peptides, a corresponding internal standard peptide, via mass spectrometry; and
(e) generating for each signature peptide, a normalized value obtained by comparing mass spectrometry signals generated by the signature peptide with mass spectrometry signals generated by the corresponding internal standard, wherein a difference between the normalized value for the signature peptide that is differentially released and a normalized value for the signature peptide that is not differentially released, or wherein a difference between the normalized value for the signature peptide that is differentially released and a control, is indicative of presence of an aberrant prion protein in the product.

35. The method of claim 34 wherein said digesting comprises (c1) contacting extracted proteins of (b) with a non-specific proteinase under conditions to allow digestion of non-core prion peptides, followed by (c2) denaturing non-specific proteinase resistant core prion peptide in the presence of a denaturing agent, followed by (c3) contacting denatured core peptide with a protease, and wherein in (e) the normalized value for the signature peptide that is differentially released is compared to a control.

36. A method of detecting an aberrant prion protein in a product of human or animal origin, comprising:
(a) obtaining a sample from a product of human or animal origin;
(b) extracting prion proteins from the sample using a chaotropic agent so as to produce denatured prion proteins;
(c) digesting the denatured prion proteins to produce a composition that contains peptide fragments of the prion proteins, wherein said fragments include signature peptides;
(d) analyzing via mass spectrometry the signature peptides and for each signature peptide, a corresponding internal standard peptide; and
(e) generating for each signature peptide, a normalized value obtained by comparing mass spectrometry signals generated by the signature peptide with mass spectrometry signals generated by the corresponding internal standard peptide, wherein a difference in the normalized value for at least one of the signature peptides compared to a control is indicative of presence of the aberrant prion protein in the product.

37. The method of claim 34 or 36 wherein the product is blood or a blood-derived factor, a commercial food product or ingredient thereof, feed, or cosmetic, nutraceutical or pharmaceutical or an ingredient of said cosmetic, nutraceutical or pharmaceutical.


SEQUENCE LISTING (3 sequences)

SEQ ID NO: 1 - protein, 264 aa, Bovine sp.
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGWGQPHGGGGWGQGGTHGQWNKPSKPKTNMKHVAGAAAAGAVVGGLGGYMLGSAMSRPLIHFGSDYEDRYYRENMHRYPNQVYYRPVDQYSNQNNFVHDCVNITVKEHTVTTTTKGENFTETDIKMMERVVEQMCITQYQRESQAYYQRGASVILFSSPPVILLISFLIFLIVG

SEQ ID NO: 2 - protein, 253 aa, Homo sapiens [sequence omitted]

SEQ ID NO: 3 - protein, 10 aa, Bovine sp.
GENFTETDIK